(19) United States
(12) Patent Application Publication
(10) Pub. No.: US 2009/0234180 A1
(43) Pub. Date: Sep. 17, 2009

(54) Mir-A-Collar

(76) Inventor: Penny Ellen Gallagher, Windsor (CA)

Correspondence Address: Penny Ellen Gallagher, 3452 Barrymore Lane, Windsor Ontario, N9C 1V1, omitted

(21) Appl. No.: 11/951,502

(22) Filed: Dec. 6, 2007

(51) Int. Cl. A61N 2/00; A61D 99/00

(52) U.S. Cl. 600/15

(57) ABSTRACT

A none invasive, drug free collar that aids in the motility of warm blooded animals. This collar consists of magnets, copper chain or wire and beads of various compounds. Copper and magnets have been used for centuries to relieve pain and inflammation. The beads are attached to the copper chain.

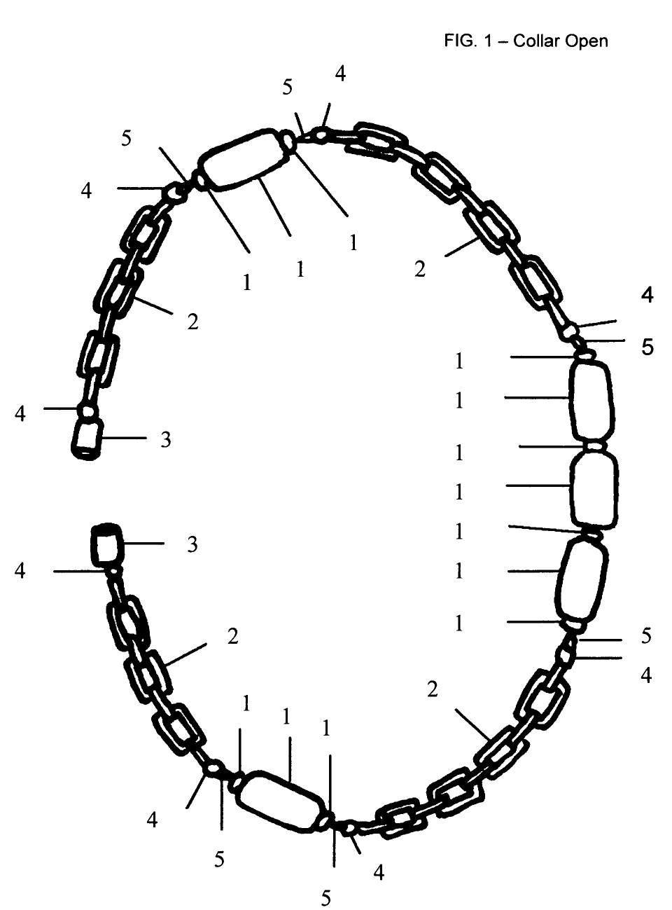

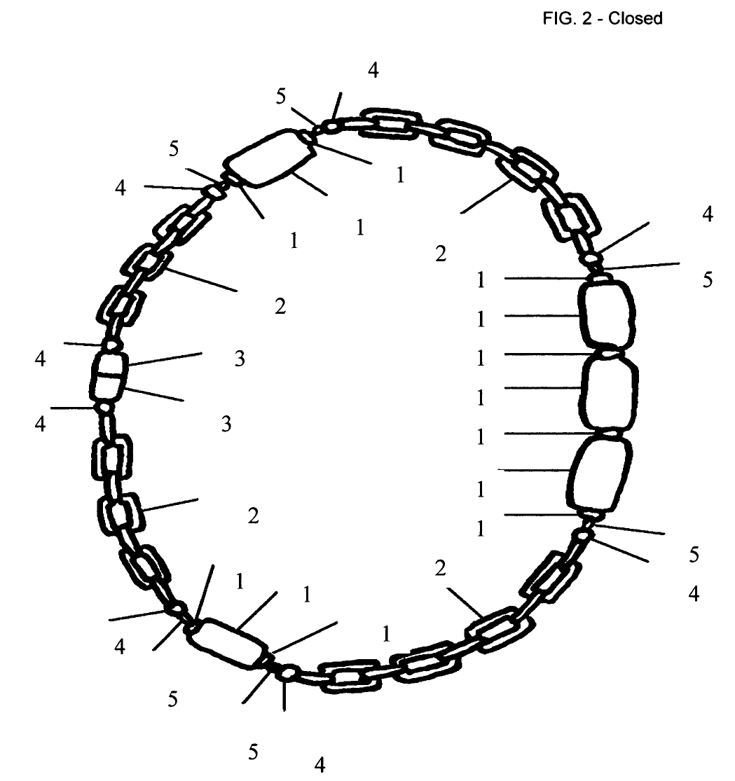

The copper chain ends fasten together with magnets. These magnets insure easy release of collar for safety to the animal but are strong enough to keep the collar secure and in place. The beads are for esthetic appeal and may consists of glass, plastic or semi precious stones including but not limited to onyx, tiger eye, jade or other precious or semi precious stone.

Sizes for beads, copper chain, wire, static magnets, and jump rings may vary according to esthetical appearance and collar size.
Collars are assembled by attaching beads (1) to the copper chain (2)
The beads are strung onto wire (5) and the wire is attached with jewellers findings (jump rings) or other appropriate connector to the copper wire/chain (4). Static magnets (3) are added to the ends of the chain for easy closure and animal safety.
The purpose of this collar is to aid in the movement of animals with motility impairments.

### CROSS REFERENCE TO RELATED APPLICATIONS

Not Applicable

### STATEMENT REGARDING FEDERALLY SPONSORED R&D

Not Applicable

### REFERENCE TO SEQUENCE LISTING

Not Applicable

### BACKGROUND

A collar designed to be worn by warm blooded mammals, as a drug free alternative, to aid in motility impairment. Collar consists of beads of various compounds, copper and magnets. Copper and magnets have been used in the homeopathic industry with great success to aid motility and ease of movement. My own case studies have involved canines, felines and humans with very satisfactory results. Both copper and magnets have been well documented as to their effectiveness.

Magnets:

Magnets produce a type of energy called magnetic fields. Magnets are widely marketed to help manage the symptoms of various diseases and conditions, including pain. Magnets used in these collars are of a type called static (or permanent) magnets, because the resulting magnetic fields are unchanging. The other magnets used for health purposes are called electromagnets, because they generate magnetic fields only when electrical current flows through them. Magnets come in different strengths, most often measured in units called gauss (G). For comparison purposes, the Earth has a magnetic field of about 0.5 G; refrigerator magnets range from 35 to 200 G; magnets marketed for the treatment of pain are usually 300 to 5,000 G; and MRI (magnetic resonance imaging) machines widely used to diagnose medical conditions non invasively produce up to 200,000 G.

The National Centre for Complimentary and Alternative Medicine does concede that scientific research to date does not firmly support a conclusion that magnets of any type can relieve pain. However, some people do experience some relief. One double blind study as published in Alternative Therapies in Health and Medicine 2002, found that when 43 participants wore knee pads containing static magnets there was significant less pain, a better daily physical function and walking speed. (Hinman et al 2002)

Another study published in Archives of Physical Medicine and Rehabilitation November 1997 documented investigators at the University of Houston who taped half-inch magnets to the sore spots of 29 people with post-polio pain and attached identical but fake magnets to a comparison group of 21 patients. Neither set of patients knew who was getting the real magnets. All the patients were asked to rate their pain on a scale of one to 10, with 10 being the most severe. Those wearing the real magnets reported a reduction in pain from a level of 9.6 to 4.4. But the 21 people treated with sham magnets said their pain dropped only from 9.9 to 8.4.

Many positive results have been documented by reputable individuals.

Copper:

Copper, a mineral, is found in trace amounts in all tissues in the body. Although only a small amount is needed, copper is an essential nutrient in the production of hemoglobin (red blood cells), myelin (the substance that surrounds nerve fibers), collagen (a component of bones and connective tissue), and melanin (a dark pigment that colors the hair and skin).

Copper can act as both an antioxidant and a pro-oxidant. As an antioxidant, it seeks out damaging particles in the body known as free radicals. Free radicals, found naturally in the body can damage cell walls, interact with genetic material, and may contribute to the aging process as well as the development of a number of health conditions. Antioxidants can neutralize free radicals and may reduce pain.

Animal studies indicate that oral copper supplements can reduce the development and progression of arthritis. Many people with arthritis (both rheumatoid and osteo) apply copper solutions to their skin or wear copper bracelets to relieve pain and inflammation of the joints. One study from the 1970s indicated that the copper bracelets worked better than placebo bracelets.

The use of copper bracelets in arthritis showed positive effects in most cases (Walker and Keats, 1976). It is interesting to note that a clinical trial of copper bracelets, by the Mayo Clinic, found that they were effective in about three quarters of the cases of musculoskeletal pain, which is roughly equivalent to the rate of effectiveness of standard drugs used to treat arthritis and other musculoskeletal pain disorders. (Bratton et al., 2002) So the only two clinical studies of copper bracelets agree that they are indeed effective in treating arthritis and musculoskeletal pain.

The copper chain or wire is available in various styles and sizes.

Beads:

Beads can be of various compounds. There is a significant following of individuals who, in their beliefs, feel some stones have specific abilities. Use of these stones could possibly add to the collar effectiveness.

### SUMMARY

The use of magnets and copper has been well documented for decades. Persons wearing these compounds claim significant reduction in pain, myself included. To date, the conventional treatment for animal arthritis has been surgery or medications. This is a medication free, non-invasive method to ease symptoms of pain in canines, felines and other warm blooded mammals. From personal experience I have seen the benefits of this collar on dogs and cats.

### BRIEF DESCRIPTION OF DRAWING VIEWS

| View 1 | Collar OPEN | View 2 | Collar CLOSED |
|---|---|---|---|
| 1 | bead | 1 | bead |
| 2 | copper chain/wire | 2 | copper chain/wire |
| 3 | static magnets | 3 | static magnets |
| 4 | jump ring | 4 | jump rings |
| 5 | wire | 5 | wire |

### DESCRIPTION OF THE PREFERRED EMBODIMENT

Collars are assembled by attaching beads (1) to the copper chain (2) The beads are strung onto wire (5) and the wire is attached with jewelers findings (ump rings) or other appropriate connector to the copper wire/chain (4). Static magnets (3) are added to the ends of the chain for easy closure and animal safety. The purpose of this collar is to aid in the movement of animals with motility impairments.

Assembly of the collar can be manually or by machine.

Previous conventional treatments for arthritis in warm blooded mammals has been surgery or medications.

The embodiments of the invention in which an exclusive property or privilege is claimed are defined as follows:

1. A collar for warm blooded mammals to aid with motility and ease of movement.

2. A collar as set out in claim 1 with the inclusion of copper chain or wire.
Wire or chain can be between 2 mm and 25 mm.

3. A collar as set out in claim 1 and claim 2 with closing apparatus of static magnets allowing for easy release for safety purposes.
Magnets may have strength of between 35 and 5,000 G.

4. A collar as set out in claim 1, claim 2 and claim 3 using beads of various compositions be it plastic, crystal, bone, metal, glass, wood, precious stones, or semi-precious stones.
Sizes may vary between 2 mm and 30 mm.

5. A collar as set out in claim 1, claim 2, claim 3 and claim 4 with the inclusion of jump rings, wire or other appropriate fastener for the purpose of connecting: chain and beads, chain and magnets, chain and wire, beads and magnets, beads and wire, magnets and wire.
